(19) United States
(12) Patent Application Publication
(10) Pub. No.: US 2023/0384190 A1
(43) Pub. Date: Nov. 30, 2023

(54) HORIZONTALLY ADJUSTABLE SAMPLE TAKER FOR DISSOLUTION APPARATUS

(71) Applicant: Agilent Technologies, Inc., Santa Clara, CA (US)

(72) Inventors: Alan Norman, Shrewsbury (GB); Jonathan Mark Barnes, Telford (GB)

(21) Appl. No.: 18/231,885

(22) Filed: Aug. 9, 2023

Related U.S. Application Data
(63) Continuation-in-part of application No. PCT/IB2022/051101, filed on Feb. 8, 2022.

(30) Foreign Application Priority Data
Feb. 9, 2021 (GB) 2101757.9

(51) Int. Cl. G01N 1/38

(52) U.S. Cl. CPC G01N 1/38; G01N 2001/386

(57) ABSTRACT

A sampling device for taking sample from a vessel of a dissolution apparatus includes a sample taker configured to be movable within a horizontal plane when mounted at the vessel.

### CROSS-REFERENCE TO RELATED APPLICATIONS

This application is a continuation-in-part of International Application No. PCT/IB2022/051101, filed on Feb. 8, 2022; which claims priority to UK Application No. GB 2101757.9, filed on Feb. 9, 2021; the entire contents of each of which are incorporated by reference herein.

### TECHNICAL FIELD

The present invention relates to a sampling device for a dissolution apparatus, a sample treatment assembly for a dissolution apparatus, a dissolution apparatus, and a method of operating a dissolution apparatus.

### BACKGROUND

Dissolution testing is often performed as part of preparing and evaluating soluble materials, particularly pharmaceutical dosage forms (for instance, tablets, capsules, and the like) consisting of a therapeutically effective amount of active drug carried by an excipient material. Typically, dosage forms are dropped into test vessels that contain dissolution media of a predetermined volume and chemical composition. For instance, the composition may have a pH factor that emulates a gastro-intestinal environment. Dissolution testing can be useful, for example, in studying the drug release characteristics of the dosage form or in evaluating the quality control of the process used in forming the dose. To ensure validation of the data generated from dissolution-related procedures, dissolution testing is often carried out according to guidelines approved or specified by certain entities such as United States Pharmacopoeia (USP), in which case the testing must be conducted within various parametric ranges. The parameters may include dissolution media temperature, the amount of allowable evaporation-related loss, and the use, position and speed of agitation devices, dosage-retention devices, and other instruments operating in the test vessel. As a dosage form is dissolving in the test vessel of a dissolution system, optics-based measurements of samples of the solution may be taken at predetermined time intervals through the operation of analytical equipment such as a spectrophotometer.

During dissolution testing, samples may be taken from a vessel by a sampling device, for instance for further analysis or documentation. However, it may be cumbersome and may be prone to errors to use a sampling device with different types of vessels.

### SUMMARY

It is an object of the present disclosure to enable sampling during dissolution testing in a flexible, simple and failure robust way.

According to an exemplary embodiment of the present disclosure, a sampling device for taking sample from a vessel of a dissolution apparatus is provided, wherein the sampling device comprises a sample taker which is configured to be movable within a horizontal plane when mounted at the vessel.

According to another exemplary embodiment of the present disclosure, a sample treatment assembly for a dissolution apparatus is provided, wherein the sample treatment assembly comprises a vessel for accommodating sample, and a sampling device having the above mentioned features and mounted at the vessel.

According to still another exemplary embodiment, a dissolution apparatus for testing dissolution of a sample is provided, wherein the dissolution apparatus comprises a sampling device having the above-mentioned features and/or a sample treatment assembly having the above mentioned features.

According to still another exemplary embodiment, a method of operating a dissolution apparatus (in particular a dissolution apparatus having the above-mentioned features) for taking sample from a vessel is provided, wherein the method comprises mounting a sampling device at the vessel accommodating the sample, and moving a sample taker of the sampling device within a horizontal plane and relative to the vessel when mounted at the vessel.

In the context of the present application, the term “dissolution apparatus” may particularly denote an apparatus configured for analyzing dissolution properties of a sample in a liquid. In particular in the pharmaceutical industry, drug dissolution testing may be used to provide in vitro drug release information for both quality control purposes, i.e. to assess batch-to-batch consistency of solid oral dosage forms such as tablets, and drug development, i.e., to predict in vivo drug release profiles. Dissolution testing may play a role in formulation decisions during product development, for equivalence decisions during generic product development, and/or for product compliance and release decisions during manufacturing.

In the context of the present application, the term “sampling device” may particularly denote a device configured for taking a sample of a substance (such as a test fluid) processed by a dissolution apparatus, wherein such a substance may be contained in a vessel of the dissolution apparatus. For the purpose of taking such a sample, the sampling device may be equipped with a sample taker, which for instance may take the sample using a cannula.

In the context of the present application, the term “sample” may particularly denote a portion of a substance processed by the dissolution apparatus, in particular being contained in a vessel thereof. For example, the sample may be a fluidic sample, i.e. may comprise a liquid and/or a gas, optionally comprising solid particles.

In the context of the present application, the term “vessel” may particularly denote a container containing a substance processed by the dissolution apparatus. The substance may be treated in the vessel in a definable way, for instance may be stirred by a stirrer immersing into the substance in the vessel, may be heated to a desired temperature, may be subject to a chemical reaction, etc.

In the context of the present application, the term “sample taker” may particularly denote a member of the sampling device being configured for actually taking the sample of the substance out of the vessel. For instance, the sample taker may comprise a movable cannula (for instance arranged at a movable cannula rack) which can be moved into the substance for drawing a sample thereof. Such a sample taker (for instance a sampling cannula) may be withdrawn once a sample has been taken, as the presence of the sample taker in the substance (such as a test fluid) may influence the hydrodynamics and may thereby influence the dissolution test.

In the context of the present application, the term “horizontal plane” may particularly denote a plane perpendicular to a central axis of a vessel, a plane perpendicular to a rotation axis of a stirrer in the vessel, and/or a plane perpendicular to an axis of gravity in a lab in which the dissolution apparatus is installed to be operative. For instance, the sample taker may be configured to freely move in the vessel within an entire two-dimensional horizontal area range. However, it may be alternatively possible that the sample taker is configured for horizontally moving in a vessel exclusively along a predefined trajectory in a guided way, in particular along a guided closed line, for instance along a guided closed circular line.

In the context of the present application, the term “vertical direction” may particularly denote a direction corresponding to a central axis of a vessel, a direction corresponding to a rotation axis of a stirrer in the vessel, and/or a direction corresponding to an axis of gravity in a lab in which the dissolution apparatus is installed to be operative.

In the context of the present application, the term “sample treatment assembly” may particularly denote an arrangement comprising at least a vessel for accommodating a substance to be treated by the dissolution apparatus, and a sampling device with sample taker assembled with, in particular mounted on, the vessel.

According to an exemplary embodiment of the present disclosure, a sampling device for a dissolution apparatus is provided, which can be flexibly used with a large variety of different vessels having different sizes, shapes and/or volumes without the need of completely changing the sampling device or disassembling and reassembling the dissolution apparatus. Moreover, a sampling device according to an exemplary embodiment of the present disclosure may also be suitable for different sample taking protocols, for instance taking sample from a specifically definable position in a vessel. Advantageously, this may be achieved by configuring the mounted sample taker to be movable within a horizontal plane relative to the vessel. Hence, the sampling device may be provided with a mechanism allowing a user to move the sample taker (preferably along a closed circular horizontal trajectory being eccentric with respect to a vessel axis) for adjusting a (preferably radial) position in a horizontal plane at which position the sample shall be taken. When for example a relatively large vessel is to be used, the mechanism may be actuated for moving the sample taker to a radially outward position. When however, for instance a relatively small vessel is to be used, the mechanism may be actuated for moving the sample taker to a radially inward position. This allows to flexibly adjust, in a simple and failure robust way, the sample taker to a desired sample taking position within a horizontal plane.

In the following, further embodiments of the sampling device, the sample treatment assembly, the dissolution apparatus, and the method will be explained.

An aspect of an exemplary embodiment of the present disclosure may be to vary the XY-position(s) of a dissolution sampling mechanism, for instance by an eccentric rotation of the sampling device or part thereof. This may allow an easy and intuitive positioning of the sampling mechanism, for example in order to comply with different standards, experimental protocols, and/or shapes and/or sizes of vessels. In such a dissolution tester, it may be advantageously possible to provide a flexible positioning of the sampling (i.e. to take samples from the dissolution vessel) allowing to automatically position the sample taker in XY direction(s). This may be achieved, for instance, by an eccentric rotation of the sample taker (for example a sampling cannula and/or a cannula rack) with respect to a vessel axis, thus allowing to assume different positions within an XY-plane, and more specifically to assume different radial positions in a vessel within an XY-plane.

In an embodiment, the sample taker is configured to be additionally movable along a vertical direction when mounted at the vessel. A vertical movability of the sample taker may allow to selectively lower the sample taker for immersing it into a substance in the vessel, or to raise the sample taker out of the substance at the end of a sampling period.

In an embodiment, the sample taker is configured to be rotatable (in particular limited to a predefined circular path eccentric with respect to a vessel axis) within a horizontal plane when mounted at the vessel. Rotation of the sample taker may be enabled over a limited angular range (for instance over an angular range of up to 180°) or over an unlimited angular range (for instance may be rotated over an angular range of more than 360°). In particular, a user may simply trigger a rotation of the sample taker in the sampling device for adjusting the sample taker's radial position in the vessel.

In an embodiment, the sample taker is configured to be rotatable about a central axis of the sampling device. In particular, the central axis of the sampling device may remain spatially fixed, while the sample taker may be rotated at or close to an exterior perimeter of the sampling device. For example, the sample taker is configured to be rotatable in a concentric way about the axis of the sampling device and in an eccentric way about the axis of the vessel.

In an embodiment, the sampling device comprises an antenna, in particular configured for wireless communication with a transponder, more particularly a radiofrequency identification (RFID) tag, of the vessel. By a wireless communication between the antenna of the sampling device and the transponder of the vessel, an identity and type of the vessel may be identified, and compliance between the vessel and the sampling device may be assessed. For example, a user may use this information for verifying whether the horizontal position of the sample taker should be adapted to an identified vessel or whether a vessel should be changed.

In an embodiment, the sampling device comprises a drive unit, in particular a motor such as an electric motor, configured for providing driving power for moving the sample taker vertically. Thus, the motion in vertical direction may be automated by such a drive unit. Since sample taking may be a frequent action during operation of a dissolution apparatus, the automation of a vertical motion of the sample taker may significantly reduce the amount of user interaction in terms of sampling. Highly advantageously, by integrating such a drive unit in the sampling device, it may be possible to individually carry out a sampling procedure with an individual sample treatment assembly without the need for sampling all sample treatment assemblies of a dissolution apparatus together. However, the sampling device may alternatively comprise a manual actuation mechanism configured for manually moving the sample taker vertically.

In an embodiment, the sampling device comprises a manual actuation element configured for being manually actuated by a user for manually moving the sample taker within the horizontal plane. While the vertical motion for sampling is a very frequent task, adjusting the horizontal position of the sample taker needs to be carried out usually less frequently, for instance only when changing vessels of a dissolution apparatus or designing a new experimental setup. This may occur typically once a month. Thus, a manual actuation mechanism for adjusting the XY-position may be fully sufficient. However, the sampling device may alternatively comprise a drive unit, for instance a motor, configured for moving the sample taker within the horizontal plane.

In an embodiment, the sampling device comprises at least one sensor configured for sensing sensor data indicative of a position and/or a motion of the sample taker. More specifically, the sampling device may for example comprise at least one sensor configured for sensing sensor data indicative of a vertical position and/or a vertical motion of the sample taker. By detecting position or motion of the sample taker, the sensor may allow to provide information at which (in particular vertical) position the sampling is carried out.

In an embodiment, the sample taker comprises at least one marker, in particular at least one slit, to be sensed by the at least one sensor as being indicative of the (in particular vertical) position and/or the motion. An optical sensor arranged next to the sample taker, for instance next to a cannula rack, may detect a light pattern when a sequence of slits passes the optical sensor.

In an embodiment, the at least one sensor is mounted on a circuit board, for instance on a PCB (printed circuit board). One or more sensors may be surface mounted on such a PCB. Such a sensor system may be easily implemented in a sampling device.

In an embodiment, the sampling device comprises a motion mechanism for vertically moving the sample taker. Such a motion mechanism may also comprise the above-described drive unit, for instance a motor. By integrating the motion mechanism for vertically moving the sample taker in the sampling device, an individual sample treatment assembly may be moved independently and separately from other sample treatment assemblies of a dissolution apparatus.

In an embodiment, the motion mechanism comprises a rack and pinion assembly for carrying out the vertical motion of the sample taker. For instance, the motion mechanism may comprise a pinion gear (in particular cooperating with a worm drive gear) for engaging an array of ribs or teeth of the sample taker (in particular of a cannula rack of the sample taker). The described motion mechanism is simple and robust and can be easily integrated in a sampling device with low space consumption and low weight.

In an embodiment, the sampling device comprises a tubular sheath, in particular comprising at least two partial shells, accommodating at least part of a motion mechanism for moving the sample taker. Thus, the external appearance of the sampling device may be essentially tubular. The provision of two partial shells, which can be closed for instance by one or more locking mechanisms, provides a protective housing for the interior constituents.

In an embodiment, the sampling device is configured for withdrawing sample from the vessel. For this purpose, the sample taker may be moved vertically to immerse into substance in a vessel. Thereafter, a negative pressure may be applied (for instance by withdrawing a piston of a syringe or by operating a peristaltic pump) for drawing sample through the sample taker.

For example, the sample taker may comprise a sampling cannula for taking sample. Such a sampling cannula may be mounted at a cannula rack which can be moved upwardly or downwardly by the above-mentioned motion mechanism. Markers at such a cannula rack may be detected optically by a sensor for monitoring or controlling the sampling process.

In an embodiment, the sample treatment assembly may comprise a stirring device for stirring sample and being mounted at the vessel. For instance, the stirring device comprises a paddle rotated by a drive unit such as an electric motor. This may properly mix a substance in the vessel.

In an embodiment, the sampling device is mounted laterally displaced with respect to a central axis of the vessel, i.e. eccentric with respect to the vessel axis. A stirring device for stirring sample in the vessel may extend along the central axis of the vessel. Sampling device and stirring device may thus be arranged to operate simultaneously without blocking each other. For example, there may be various positions in relation to a vessel axis and fluid height in which regulatory requirements state that samples shall be taken. Consequently, it may be possible to properly stir the substance in the vessel by a central stirring device without undesired interaction with a sampling process, when the sampling device is laterally displaced with respect to a vessel axis. With such a configuration, rotation of the sample taker changes a radial distance of the sample taker with respect to the central axis of the vessel. Thus, the described design makes it possible to adapt a sampling device to a specific vessel by a mere rotation of the sample taker relative to the vessel and relative to a central axis of the vessel to adapt the radial sample taking position.

In an embodiment, the sample treatment assembly comprises a vessel cover member covering an opening of the vessel and accommodating the sampling device in a horizontally rotatable way. The vessel cover member may be a lid member for closing an opening of the vessel containing the substance to be analysed. Moreover, the vessel cover member may have a mounting opening for mounting the sampling device, preferably displaced with regard to a central axis of the vessel. With such a configuration, rotation of the sample taker changes a radial distance of the sample taker with respect to the central axis of the vessel.

In an embodiment, the dissolution apparatus comprises a plurality of sampling devices and/or sample treatment assemblies, for instance having the features as described above. For example, a number of sample treatment assemblies of a dissolution apparatus may be in a range from 2 to 20, in particular from 4 to 15, for example 8. This may allow to carry out a dissolution analysis, to stir substance in vessels, and to carry out sampling the vessels on a large scale.

In an embodiment, the sample taker of the sampling device is configured to be rotated about a sampling device axis parallel to a central axis of the vessel, and rotation of the sample taker changes a radial distance of the sample taker with respect to the central axis of the vessel.

In an embodiment, the sampling device comprises a holding mechanism configured to support the sample taker and accommodate rotation of the sample taker about the sampling device axis.

In an embodiment, the holding mechanism comprises an actuation element to which the sample taker is mounted, wherein actuation of the actuation element rotates the sample taker about the sampling device axis.

In an embodiment, the sampling device comprises a holding mechanism configured to be movable along a vertical direction, wherein the sample taker is movable with the holding mechanism along the vertical direction.

In an embodiment, the sampling device comprises a holding mechanism configured to support the sample taker and accommodate rotation of the sample taker about the sampling device axis. and the holding mechanism is also configured to be movable along a vertical direction, wherein the sample taker is movable with the holding mechanism along the vertical direction.

According to another embodiment, a sample treatment assembly for a dissolution apparatus comprises: a sampling device according to any of the embodiments disclosed herein; and a vessel cover member configured to be mounted to an opening of the vessel, wherein the vessel cover member comprises an arcuate aperture through which the sample taker is extendable into the vessel when the sample taker is mounted at the vessel, and the sample taker is movable within the arcuate aperture in the horizontal plane while being rotated about the sampling device axis.

In an embodiment, the sample treatment assembly comprises a pillar mounted to or integrated with the vessel cover and arranged along a pillar axis, wherein the pillar axis corresponds to the sampling device axis and the sample taker is rotatable around the pillar.

In an embodiment, the sample treatment assembly comprises a holding mechanism configured to support the sample taker and accommodate rotation of the sample taker around the pillar, and/or configured to be movable along a vertical direction relative to the pillar, wherein the sample taker is movable with the holding mechanism along the vertical direction.

According to another embodiment, a sample treatment assembly for a dissolution apparatus comprises: a plurality of sampling devices according to any of the embodiments described herein, the plurality of sampling devices comprising a plurality of sample takers, each sample taker associated with a corresponding one of the sampling devices, wherein the sampling devices are configured to be respectively mounted at a plurality of vessels; and a holding mechanism comprising a plurality of actuation elements to which the sample takers are respectively mounted, wherein each actuation element is configured to rotate a corresponding one of the sample takers within the horizontal plane.

In an embodiment, the holding mechanism is configured to rotate each sample taker, via a corresponding one of the plurality of actuation elements, independently of other ones of the plurality of sample takers, or to rotate each sample taker together with one or more of the other ones of the plurality of sample takers.

In an embodiment, the holding mechanism is configured to move the sample takers along a vertical direction.

In an embodiment, the holding mechanism is configured to move each sample taker along the vertical direction independently of other ones of the plurality of sample takers, or together with one or more of the other ones of the plurality of sample takers.

Preferably, at least one of the aforementioned sampling devices comprises a sample taker which is configured to be movable within the horizontal plane and/or vertically independently of a sample taker of at least one other of the sampling devices. This may be accomplished by providing a vertical motion mechanism and/or a horizontal motion mechanism individually and separately in each sample treatment assembly (rather than providing a uniform manifold being only movable together for all sample treatment assemblies).

### BRIEF DESCRIPTION OF THE DRAWINGS

Other objects and many of the attendant advantages of embodiments of the present disclosure will be readily appreciated and become better understood by reference to the following more detailed description of embodiments in connection with the accompanying drawings. Features that are substantially or functionally equal or similar will be referred to by the same reference signs.

FIG. 1 shows a dissolution apparatus in accordance with embodiments of the present disclosure.

FIG. 2 illustrates an exploded view of part of a sampling device according to an exemplary embodiment of the present disclosure.

FIG. 3 illustrates a partially assembled view of the part of the sampling device according to FIG. 2.

FIG. 4 illustrates a partially assembled view of the part of the sampling device according to FIG. 3 in a tubular sheath.

FIG. 5 illustrates an exploded view of constituents of a motion mechanism for vertically moving a sample taker of the sampling device according to FIG. 2 to FIG. 4.

FIG. 6 illustrates the constituents of the motion mechanism according to FIG. 5 in a partially assembled state.

FIG. 7 illustrates a three-dimensional view of a sampling device with the constituents according to FIG. 2 to FIG. 6 in an assembled state.

FIG. 8 illustrates another three-dimensional view of the sampling device of FIG. 7.

FIG. 9 illustrates a plan view of a sample treatment assembly with a sampling device according to an exemplary embodiment of the present disclosure in a first operation state.

FIG. 10 illustrates a plan view of the sample treatment assembly of FIG. 9 in a second operation state.

FIG. 11 illustrates a three-dimensional view of a sample treatment assembly with a sampling device according to an exemplary embodiment of the present disclosure in a first operation state.

FIG. 12 illustrates a three-dimensional view of the sample treatment assembly of FIG. 11 in a second operation state.

FIG. 13 illustrates a cross-sectional view of a sampling device according to an exemplary embodiment of the present disclosure.

FIG. 14 illustrates a three-dimensional view of the sampling device of FIG. 13.

FIG. 15 illustrates another cross-sectional view of the sampling device of FIG. 13 and FIG. 14.

FIG. 16 illustrates a plan view of the sampling device of FIG. 13 to FIG. 15.

FIG. 17 illustrates a transparent plan view of the sampling device of FIG. 13 to FIG. 16.

FIG. 18 illustrates a three-dimensional view of a sample treatment assembly according to an exemplary embodiment of the present disclosure.

FIG. 19 illustrates a side view of the sample treatment assembly according to FIG. 18.

FIG. 20 illustrates a detail of the sample treatment assembly according to FIG. 18 and FIG. 19.

FIG. 21 illustrates a plan view of a sample treatment assembly with another vessel than in FIG. 18 according to an exemplary embodiment of the present disclosure.

FIG. 22 illustrates a detail of the sample treatment assembly according to FIG. 21.

FIG. 23 illustrates a plan view of the sample treatment assembly according to FIG. 21 and FIG. 22.

FIG. 24 illustrates a plan view of a sample treatment assembly with a sampling device according to an exemplary embodiment of the present disclosure.

FIG. 25 illustrates a top perspective view of a sample treatment assembly with a sampling device according to an exemplary embodiment of the present disclosure.

FIG. 26 illustrates a bottom perspective view of the sample treatment assembly according to FIG. 25.

FIG. 27 illustrates a top plan view of the sample treatment assembly according to FIG. 25.

FIG. 28 illustrates a bottom top plan view of the sample treatment assembly according to FIG. 25.

FIG. 29 illustrates a front plan view of the sample treatment assembly according to FIG. 25.

FIG. 30 illustrates an end plan view of the sample treatment assembly according to FIG. 25.

The illustrations in the drawings are schematic.

### DETAILED DESCRIPTION

Before describing the figures in further detail, some basic considerations of the present disclosure will be summarized based on which exemplary embodiments have been developed.

In a conventional dissolution apparatus, automated dissolution sampling is supported. With such a conventional tester sampling system it may be possible to introduce a sampling cannula into (for example eight) test vessels so that a sample of test fluid can be pumped out via an automated pumping system or a manually operated syringe. A sampling cannula can be withdrawn once the sample has been taken. This allows a conventional dissolution apparatus to insert and withdraw sample cannulas at the required time points and meet regulatory height requirements.

There may be various positions in relation to a vessel axis and fluid height in which regulatory requirements state that samples shall be taken. In order to meet relative vessel axis position requirements, the cannulas are conventionally mounted into pre-set positions on the manifold. Conventionally, it may for instance be possible to use such a manifold to lower all its sampling cannulas altogether. However, any change in the vessel axis position requirement requires to partially disassemble the sampling system. Such a conventional sampling system also requires that all test vessels in the dissolution tester have identical height requirements and simultaneous sampling times.

Another conventional approach negates the requirement for simultaneous sampling times, but it does not allow for any deviation in the relative vessel axis position requirement. Said other conventional approach is relatively complex and requires a high number of parts, which is detrimental to its manufacturability and to the ease-of-use for a user without specific skills.

In order to overcome at least part of the above-mentioned and/or other shortcomings of conventional approaches, an exemplary embodiment of the present disclosure provides a sampling device for taking a sample of a substance (such as a test fluid) from a vessel using a sample taker being configured to be movable within a horizontal plane relative to a vessel at which the sampling device may be mounted. More specifically, an exemplary embodiment of the present disclosure may enable the user to vary the XY position of a sample taker by rotating it relative to a vessel (in particular by rotating it along a circular trajectory) on which the sampling device is mounted. This may make it possible to provide a sampling device for a dissolution apparatus meeting different standards defining at which position sampling has to be carried out relative to stirring in a vessel. In particular, a sampling device according to exemplary embodiments of the present disclosure may make it possible to change XY-coordinates of a sample taker without disassembly of a sample treatment assembly or even an entire dissolution apparatus.

Advantageously, an exemplary embodiment of the present disclosure may provide a modular sampling mechanism being simple in manufacture and versatile in use and allowing to fulfill regulatory requirements of dissolution sampling. Moreover, an embodiment may also have a facility to detect and identify a vessel (for instance for determining a vessel type), for example by equipping the sampling device with an antenna and the vessel with a transponder for wireless communication with the antenna.

According to an exemplary embodiment of the present disclosure, a dissolution apparatus with modular sampling system is provided which may be able to meet a large variety of height and relative vessel axis positional requirements without disassembly. For instance, sample takers (which may comprise sampling cannulas) of different sample treatment assemblies of a dissolution apparatus can be individually driven and manipulated.

Construction of a sampling device, a sample treatment assembly and a dissolution apparatus according to exemplary embodiments of the present disclosure may be significantly simplified compared with conventional approaches. This allows the manufacture of the system with reasonable effort. Additionally, a method according to an exemplary embodiment of the present disclosure which may be used to position a sample taker (such as a cannula) relative to a vessel axis may allow the sample mechanism to utilize an antenna to identify various types of (for instance RFID tagged) vessels in close proximity. This allows for verification of conformity to required test conditions.

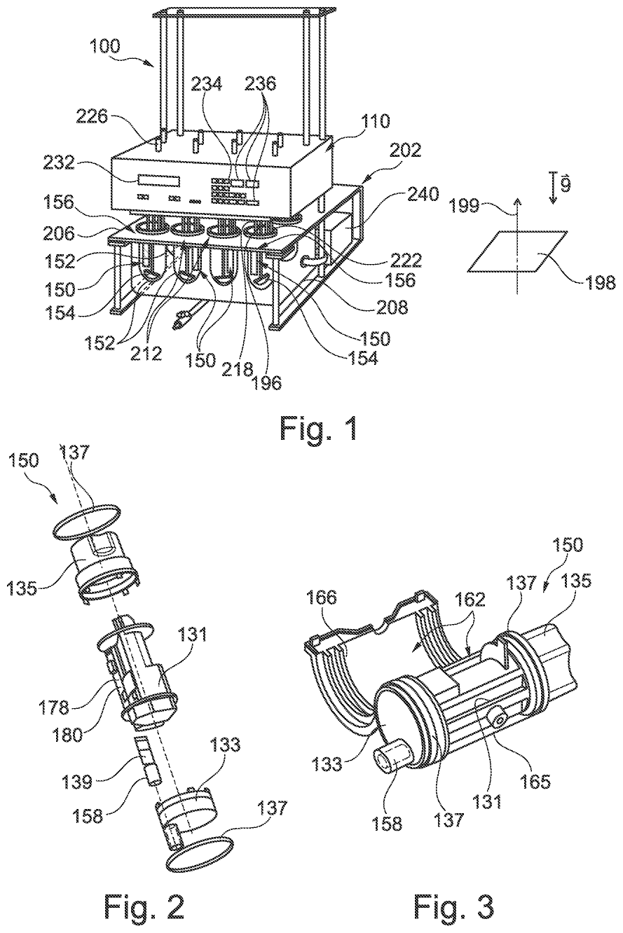

FIG. 1 is a perspective view of an example of a dissolution apparatus 100 (which may also be denoted as a dissolution test apparatus) according to an exemplary embodiment of the present disclosure.

The dissolution apparatus 100 may include a frame assembly 202 supporting various components such as a main unit 110, a vessel support member 206 (for instance, a plate, etc.) below the main unit 110, and a water bath container 208 below the vessel support member 206. The vessel support member 206 supports a plurality of vessels 152 extending into the interior of the water bath container 208 at a plurality of vessel mounting sites 212. FIG. 1 illustrates eight vessels 152 by example, but it will be understood that more or less vessels 152 may be provided. Vessel covers (not shown in FIG. 1) may be provided to prevent loss of media from the vessels 152 due to evaporation, volatility, etc. Water or other suitable heat-carrying liquid medium may be heated and circulated through the water bath container 208 for example by an external heater and pump module 240, which may be included as part of the dissolution apparatus 100.

The main unit 110 of the dissolution apparatus 100 may include mechanisms for operating or controlling various components that operate in the vessels 152. For example, the main unit 110 may support stirring devices 154 having paddles operating in each vessel 152. The main unit 110 also includes mechanisms for driving the rotation of the stirring devices 154.

Moreover, media transport cannulas that provide liquid flow paths between liquid lines and corresponding vessels 152 may be operated and controlled. The media transport cannulas may include media dispensing cannulas 218 for dispensing media into the vessels 152 and media aspirating cannulas 196 of a schematically illustrated sampling device 150 for removing media, a substance or a sample (such as a test fluid) from the vessels 152. The main unit 110 may include mechanisms for operating or controlling other types of in situ operative components 222 such as fiber-optic probes for measuring analyte concentration, pH detectors, dosage form holders (for instance, USP-type apparatus such as baskets, nets, cylinders, etc.), video cameras, etc. A dosage delivery module 226 may be utilized to preload and drop dosage units (for instance, tablets, capsules, or the like) into selected vessels 152 at prescribed times and media temperatures.

The main unit 110 may include a programmable systems control module for controlling the operations of various components of the dissolution apparatus 100 such as those described above. Peripheral elements may be located on the main unit 110 such as an LCD display 232 for providing menus, status and other information; a keypad 234 for providing user-inputted operation and control of spindle speed, temperature, test start time, test duration and the like; and readouts 236 for displaying information such as rounds per minute, temperature, elapsed run time, vessel weight and/or volume, or the like.

The media dispensing cannulas 218 and the media aspirating cannulas 196 may communicate with a pump assembly (not shown) via fluid lines (for instance, conduits, tubing, etc.). The pump assembly may be provided in the main unit 110 or as a separate module supported elsewhere by the frame 202 of the dissolution apparatus 100, or as a separate module located external to the frame 202. The pump assembly may include separate pumps for each media dispensing line and/or for each media aspirating line. The pumps may be of any suitable design, one example being the peristaltic type. The media dispensing cannulas 218 and the media aspirating cannulas 196 may constitute the distal end sections of corresponding fluid lines and may have any suitable configuration for dispensing or aspirating liquid (for instance, tubes, hollow probes, nozzles, etc.).

In a typical operation, each vessel 152 is filled with a predetermined volume (for instance 1 liter or 250 ml) of dissolution media by pumping media to the media dispensing cannulas 218 from a suitable media reservoir or other source (not shown). One of the vessels 152 may be utilized as a blank vessel and another as a standard vessel in accordance with dissolution testing procedures to be carried out. Dosage units are dropped into one or more selected media-containing vessels 152, and each stirring device 154 is rotated within its vessel 152 at a predetermined rate and duration within the test solution as the dosage units dissolve. In other types of tests, a cylindrical basket or cylinder (not shown) loaded with a dosage unit is assembled with a respective vessel 152 and rotates or reciprocates within the test solution. For any given vessel 152, the temperature of the media may be maintained at a prescribed temperature (for instance, approximately 37±0.5° C.). The mixing speed of the stirring device 154 may also be maintained for similar purposes. Media temperature is maintained by immersion of each vessel 152 in the water bath of water bath container 208, or alternatively by direct heating. The various operative components 150, 154, 218, 196, 222 provided may operate continuously in the vessels 152 during test runs. Alternatively, the operative components 150, 154, 218, 196, 222 may be lowered manually or by an automated assembly into the corresponding vessels 152, left to remain in the vessels 152 only while sample measurements are being taken at allotted times, and at all other times kept outside of the media contained in the vessels 152. In some implementations, submerging the operative components 150, 154, 218, 196, 222 in the vessel media at intervals may reduce adverse effects attributed to the presence of the operative components 150, 154, 218, 196, 222 within the vessels 152.

During a dissolution test, sample aliquots of media may be pumped from the vessels 152 via the media aspiration cannulas 196 and conducted to an analyzing device (not shown) such as, for example, a spectrophotometer to measure analyte concentration from which dissolution rate data may be generated. In some procedures, the samples taken from the vessels 152 are then returned to the vessels 152 via the media dispensing cannulas 218 or separate media return conduits. It is also possible that a sample concentration may be measured directly in the vessels 152 by providing fiber-optic probes. After a dissolution test is completed, the media contained in the vessels 152 may be removed via the media aspiration cannulas 196 or separate media removal conduits.

According to an exemplary embodiment of the present disclosure, a respective sampling device 150 for taking sample from an assigned vessel 152 of the dissolution apparatus 100 comprises a sample taker 190 (see for instance FIG. 7 and FIG. 8) which is configured to be movable within a horizontal plane 198 when mounted at the vessel 152. As shown in FIG. 1, horizontal plane 198 may be oriented perpendicular to a vertical direction 199, the latter corresponding to or being parallel to the direction of the force of gravity (see the g-vector in FIG. 1).

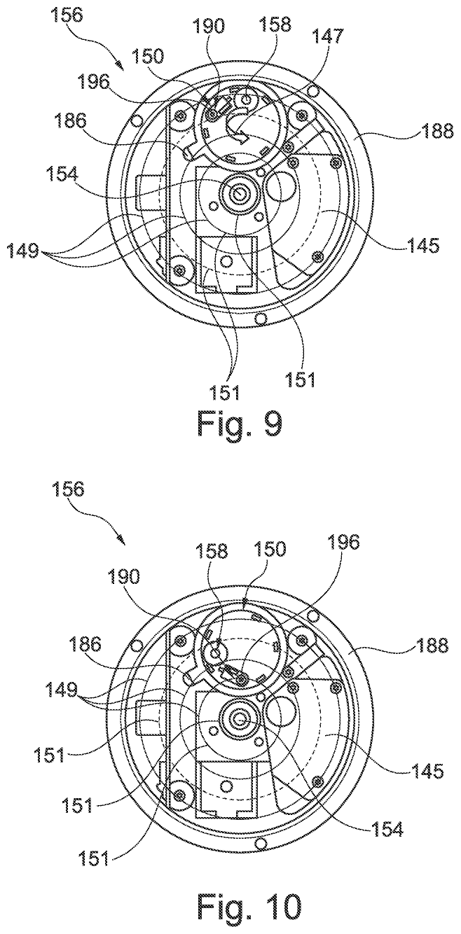

Still referring to FIG. 1, each set of vessel 152 and assigned sampling device 150 may constitute a respective sample treatment assembly 156, as shown in detail for instance in FIG. 9 to FIG. 12. In the embodiment of FIG. 1, eight sample treatment assemblies 156 may be provided, each of which being controllable individually in terms of sampling, and in particular in terms of moving in the horizontal plane 198 and/or along the vertical direction 199. In particular, each of the sampling devices 150 may comprise a sample taker 190 which may be configured to be movable, when mounted at a vessel 152, within the horizontal plane 198 and/or along the vertical direction 199 independently of the sample takers 190 of the other sampling devices 150.

Referring to FIG. 2 to FIG. 30, different embodiments of sampling devices 150 and sample treatment assemblies 156 according to exemplary embodiments are shown, which may be implemented for instance in the dissolution apparatus 100 according to FIG. 1, or into any other dissolution apparatus.

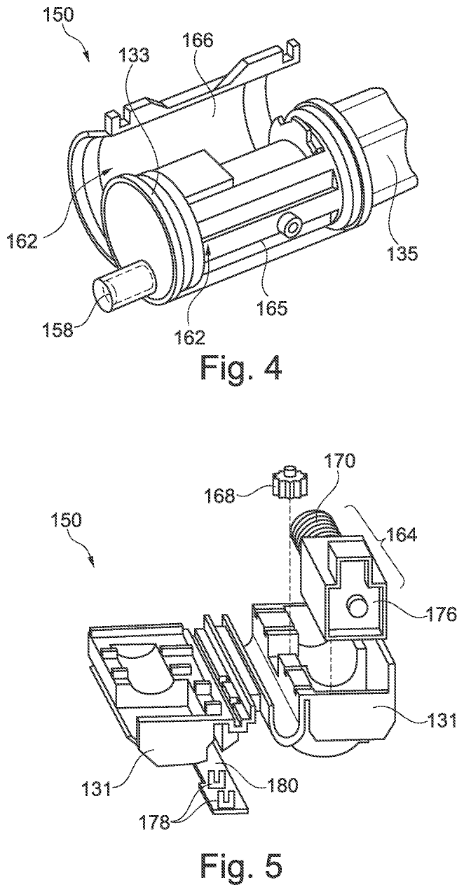

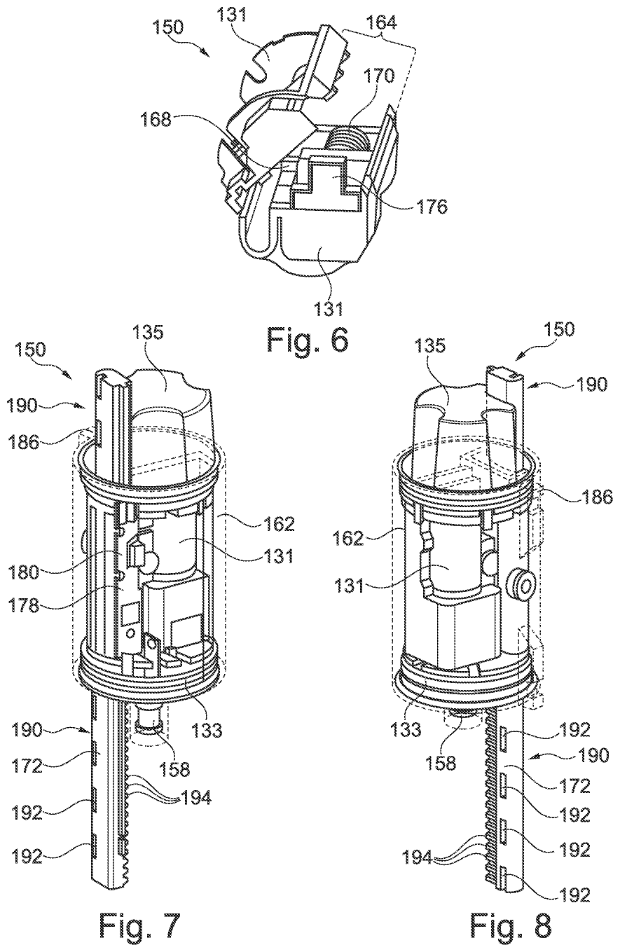

FIG. 2 illustrates an exploded view of part of a sampling device 150 according to an exemplary embodiment of the present disclosure. FIG. 3 illustrates a partially assembled view of the part of the sampling device 150 according to FIG. 2. FIG. 4 illustrates a partially assembled view of the part of the sampling device 150 according to FIG. 3 in a tubular sheath 162. FIG. 5 illustrates an exploded view of constituents of a motion mechanism 164 for vertically moving a sample taker 190 of the sampling device 150 according to FIG. 2 to FIG. 4. FIG. 6 illustrates the constituents of the motion mechanism according to FIG. 5 in a partially assembled state. FIG. 7 illustrates a three-dimensional view of a sampling device 150 with the constituents according to FIG. 2 to FIG. 6 in an assembled state. FIG. 8 illustrates another three-dimensional view of the sampling device 150 of FIG. 7.

As already mentioned, and as best seen in FIG. 7 and FIG. 8, the sampling device 150 may comprise a sample taker 190 having a cannula rack 172 which may be configured to be movable, when mounted at a vessel 152, within the horizontal plane 198 (compare the positions of sample taker 190 in FIG. 7 and FIG. 8). Moreover, the illustrated sample taker 190 is configured to be movable along a vertical direction 199 when mounted at the vessel 152. By the vertical movability of the sample taker 190, it may be possible to selectively immerse the sample taker 190 into a substance or a medium (such as a sample fluid) contained in vessel 152 during execution of sampling and to withdraw the sample taker 190 for enabling undisturbed stirring of the substance or medium before and after sampling. This may be accomplished by moving cannula rack 172 of sample taker 190 in a vertical direction 199, while a tubular sheath 162 housing several constituents of the sampling device 150 remains at a vertically fixed position relative to a vessel 152.

More specifically, the sample taker 190 is configured to be rotatable about an axis of the sampling device 150 and within a horizontal plane 198 when mounted at the vessel 152. Descriptively speaking, the sample taker 190 is configured to be rotatable in a concentric way about the axis of the sampling device 150 and in an eccentric way about the axis of the vessel 152. This can be taken for instance from a comparison of the side views of FIG. 7 and FIG. 8, and also in the top views of FIG. 9 and FIG. 10. By adjusting the position of the sample taker 190 in the horizontal plane 198 relative to vessel 152, it may be possible to use the sampling device 150 with very different vessels 152 having different dimensions and/or shapes without disassembly of the dissolution apparatus 100. Such a horizontal positioning provision of sample taker 190 may also allow to comply with different specifications in terms of a position of sample taker 190 in a vessel 152 at which a sample shall be taken in accordance with a predefined dissolution test protocol (which may be defined by an official authority).

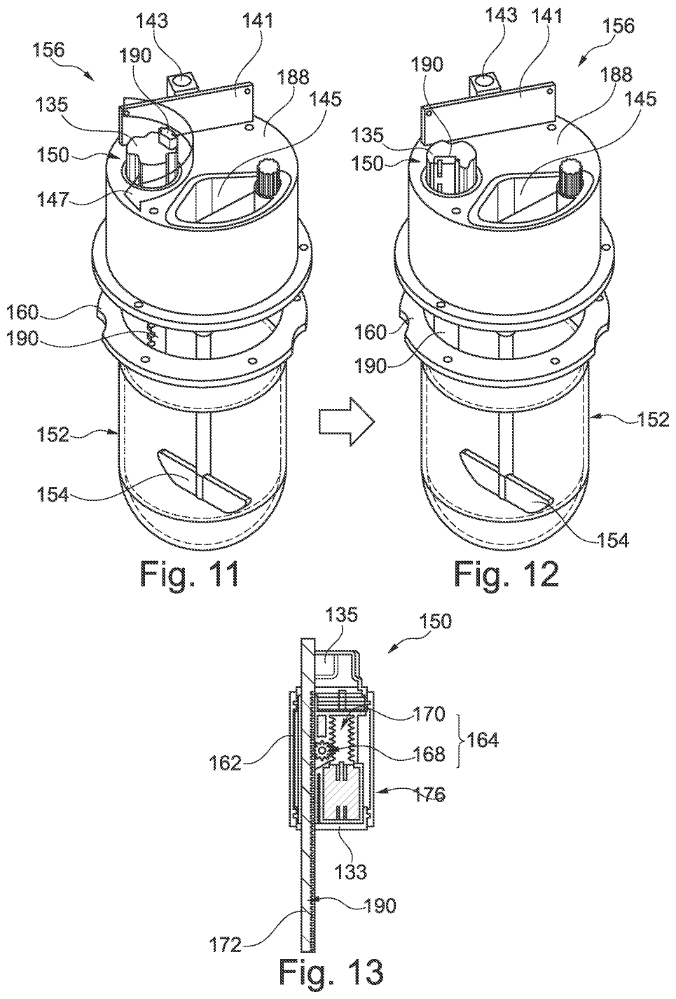

Now referring to FIG. 2 to FIG. 4 and FIG. 7 and FIG. 8, the sampling device 150 may comprise an antenna 158 being configured for wireless communication with a transponder 160, such as a radiofrequency identification (RFID) tag, of the vessel 152 (for instance arranged at a rim of a vessel 152). Said transponder 160 may be positioned for example as shown in FIG. 11 and FIG. 12 and may allow the sampling device 150 to determine an identity or type of a vessel 152 at which the sampling device 150 is mounted. By the communication between the transponder 160 and the antenna 158, a correct combination of sampling device 150 and vessel 152 can be ensured. If the determination provides the result that the combination is incorrect, a user may be informed about this fact.

FIG. 5 and FIG. 6 illustrate that the sampling device 150 comprises a drive unit 176 embodied as an electric motor and configured for providing driving power for moving the sample taker 190 vertically. Drive unit 176 may be provided with electric energy by a wiring connection (not shown). Drive unit 176 forms part of a motion mechanism 164 for vertically moving the sample taker 190. Said motion mechanism 164 comprises a rack and pinion assembly: A pinion gear 168 cooperates with a worm drive gear 170 for motion along an array of ribs 194 of cannula rack 172 of the sample taker 190 (see also FIG. 7 and FIG. 8). Thus, the frequent task of sampling by lowering the sample taker 190 into substance in vessel 152, withdrawing sample from the vessel 152 and again raising the sample taker 190 out of the substance in the vessel 152 to not disturb the dissolution process may be carried out in automated way with drive force provided by drive unit 176.

Furthermore, the sampling device 150 comprises a manual actuation element 135 configured for being manually actuated by a user for purely manually moving the sample taker 190 within the horizontal plane 198. This is shown for instance in FIG. 7 and FIG. 8. Manual actuation element 135 may be manipulated to rotate (for example between thumb and two fingers of a user) the internal elements of the sampling device 150 with the shell or tubular sheath 162. Adapting the angular position of the sample taker 190 with respect to a center of the sampling device 150 and simultaneously adapting the radial position of the sample taker 190 relative to the vessel 152 may be a task which is usually carried out significantly less frequently than sampling test fluid, for instance only when changing a vessel configuration (for example typically once a month). Thus, the adjustment mechanism for adapting a horizontal position of the sample taker 190 relative to the vessel 150 may be a manual mechanism to be carried out by a user by gripping and rotating manual actuation element 135 over a desired angular range. By such an actuation, the user may rotate the sample taker 190 for instance between the configurations shown in FIG. 7 and FIG. 8. Still referring to FIG. 7 and FIG. 8 and additionally to FIG. 9 and FIG. 10, a part 186 (which may be denoted as housing shell tang) may be used to fix the angular position of the shell or tubular sheath 162 of the sampling mechanism 150 in vessel cover member 188 (which may also be denoted as smart head).

Although not shown, one or more further markers may be provided which indicate to a user how long to turn the sample taker 190 to reach a desired position in the horizontal plane 198. It may also be possible to detect such a marker by a further (for instance magnetic) sensor to provide a user with a (for instance optical and/or acoustic) feedback when a desired horizontal position has been reached. It may also be possible that the user receives a haptic feedback when reaching a desired position. This may ensure a user-friendly and failure robust operation of the sampling device 150.

As shown in FIG. 2, FIG. 5 and FIG. 7, the sampling device 150 furthermore comprises one or more optical sensors 178 configured for sensing sensor data indicative of a vertical position and/or a vertical motion of cannula rack 172 of the sample taker 190. Correspondingly, the sample taker 190 comprises optically detectable markers in form of slits 192 in the cannula rack 172 which can be sensed optically by the one or more optical sensors 178. Captured detection signals may be evaluated by a processor (not shown) for deriving information concerning the vertical position and/or motion of the cannula rack 172 of the sample taker 190. As shown, the one or more optical sensors 178 may be mounted on a printed circuit board 180. Sensor monitoring of the vertical position of the cannula rack 172 may ensure that the sample taker 190 is always at a correct position in vertical direction 199.

FIG. 3 shows that the sampling device 190 comprises a tubular sheath 162 as an exterior casing. In the shown embodiment, tubular sheath 162 is composed of two partial shells 165, 166 which accommodate the above described motion mechanism 164 for moving the sample taker 190. The various constituents of the sampling device 190 may thus be mechanically protected in an interior of the robust sheath 162.

As already mentioned, the sample taker 190 is configured for withdrawing sample from the vessel 152 when the sample taker 190 is vertically moved into test fluid in the vessel 152 and a negative pressure is applied to media aspirating or sampling cannula 196 of the sample taker 190. For instance, the withdrawal force may be provided by a (for instance manually operated) syringe or a (for instance automatically controlled) peristaltic pump.

FIG. 5 illustrates a casing 131 having different accommodation recesses for accommodating several constituents of the motion mechanism 164. In the shown embodiment, casing 131 is a two-piece casing, and the two connected parts or sub-shells of casing 131 have a clam shell shape. As shown, casing 131 may be populated with the motor-type drive unit 176, opto-sensor printed circuit board 180 with assembled sensor(s) 178, pinion gear 168, and worm drive gear 170. FIG. 6 shows that casing 131, which is shown in the open state in FIG. 5, is then closed. Wiring (not shown) may be guided into casing 131, for instance through fairleads in an upper stiffener member. The design of casing 131 ensures a failure robust accommodation of its constituents which are also mechanically protected in a reliable way.

FIG. 2 shows casing 131 in its closed state together with a lower cap 133, an upper cap which functions as manual actuation element 135, and O-rings 137. As shown, antenna 158 (which may be mounted on a further circuit board 139) may be fitted in lower cap 133. The lower cap 133 and the upper cap may then be clipped into position. The O-rings 137 (for instance made of PTFE, polytetrafluoroethylene) may then be fitted at an upper and a lower position.

Now referring to FIG. 3, the constituents of FIG. 2 have been assembled and are now placed into tubular sheath 162. A grommet may be fitted over the wiring (not shown) and into a recess. The tubular sheath 162 is then closed, see FIG. 4. When casing 131 and tubular sheath 162 are closed, it may be ensured that the wiring remains in a static state and is not stretched.

FIG. 9 illustrates a plan view of a sample treatment assembly 156 with a sampling device 150 (in particular one of the type according to FIG. 2 to FIG. 8) according to an exemplary embodiment of the present disclosure in a first operation state. FIG. 10 illustrates a plan view of the sample treatment assembly 156 of FIG. 9 in a second operation state. FIG. 11 illustrates a three-dimensional view of a sample treatment assembly 156 with a sampling device 150 according to an exemplary embodiment of the present disclosure in a first operation state. FIG. 12 illustrates a three-dimensional view of the sample treatment assembly 156 of FIG. 11 in a second operation state.

Each of sample treatment assemblies 156 for a dissolution apparatus 100 may comprise a vessel 152 for accommodating sample or test fluid and a sampling device 150 mounted at the vessel 152. As shown in FIG. 11 and FIG. 12, the sampling device 150 is mounted with its axis being laterally displaced with respect to a central axis of the vessel 152. A stirring device 154 is also illustrated and is configured for stirring sample in the vessel 152. Stirring device 154 is mounted at a vessel cover member 188 which is mounted, in turn, on the vessel 152. As shown, the stirring device 154 comprises a rotating paddle which stirs and mixes the test fluid. While the stirring device 154 extends along a central axis of the vessel 152, the sampling device 150 and its sample taker 190 have a central axis parallel to the central axis of the vessel 152 but being laterally displaced with regard to the central axis of the vessel 152. With such a configuration, rotation of the sample taker 190 changes a radial distance of the sample taker 190 with respect to the central axis of the vessel 152.

As can be taken from FIG. 11 and FIG. 12, the vessel cover member 188 covers an opening of the vessel 152 and accommodates the sampling device 150 in a mounting recess and in a horizontally rotatable way. The design of vessel cover member 188 with its mounting recesses ensures that stirring device 154 and sampling device 150 are operable without undesired interaction.

During operating the dissolution apparatus 100 for taking sample from the vessel 152, the sampling device 150 has to be mounted at the vessel 152 accommodating the sample, as shown in FIG. 9 to FIG. 12. The sample taker 190 of the sampling device 150 may then be rotated within the horizontal plane 198 and relative to the vessel 152 to adjust a desired rotational and thus radial position in relation to the vessel 152. This rotation is indicated by an arrow 147 in FIG. 9 and FIG. 11.

Thus, FIG. 2 to FIG. 12 illustrate a modular dissolution sampling mechanism in which the horizontal position of a sample taker 190 can be flexibly adjusted to the needs of a specific vessel 152 and/or to the needs of a dissolution test protocol or specification without disassembling the dissolution apparatus 100.

It can be seen from the assembly diagrams that the core of the sampling device 150 or sample mechanism assembly is free to rotate within tubular sheath 162. Descriptively speaking, tubular sheath 162 may be shaped similarly to a clam shell. The described action facilitates the adjustment of the sample position, more precisely the position of the sample taker 190, relative to the central axis of the vessel 152 and also facilitates the adjustment of the detection area of the antenna 158.

Now referring in detail to FIG. 9 and FIG. 10, arrow 147 indicates that the sample mechanism can be rotated by the operator to adjust the sampling point to the required diameter. Solid concentric lines 149 represent the internal diameters of various vessel types. Dashed concentric lines 151 represent the required sampling diameter of various vessel types. In addition, as the sampling position of the sample taker 190 is adjusted to suit the presently used vessel 152, the position of the RFID antenna 158 may also be adjusted to align with, and to detect, an RFID tag or another transponder 160 at the rim of the vessel 152. The ease and simplicity of the positional adjustment in the horizontal plane 198 significantly improves flexibility and failure robustness of the sampling device 150 compared with conventional approaches.

Again referring to FIG. 9 and FIG. 10, sample taker 190 moves in a motor driven way perpendicular to the paper plane of FIG. 9 and FIG. 10 before and after taking a sample. For adjusting a radial position of sample taker 190 relative to a vessel 152, sample taker 190 is rotated, for instance between the two different radial positions according to FIG. 9 and FIG. 10. For this purpose, eccentric sample taker 190 can be operated by hand for changing its radial position in relation to vessel 152. The configuration of the sampling device 150 may be so that all functional parts within an outer shell of the sampling device 150 may be turned by hand.

FIG. 11 and FIG. 12 illustrate a further circuit board 141 (such as a further PCB) mounted on a top of vessel cover member 188. A controller chip for controlling operation of the sample treatment assembly 156 may for instance be mounted on the further circuit board 141. For example, the controller chip may turn the stirring device 154 on or off. Furthermore, a cable adapter 143 is mounted on top of vessel cover member 188 for providing a cable connection between the sample treatment assembly 156 and a main unit 110 of a dissolution apparatus 100. By said cable connection, electric power and communication signals may be transmitted. Still referring to FIG. 11 and FIG. 12, a supply member 145 is arranged in the vessel cover member 188 for supplying a medium (for instance a tablet or pill) to the vessel 152. For this purpose, a flap of the supply member 145 may be opened, and the media may be dropped inside the vessel 152. Thereafter, the flap may be closed again. Although not shown, the illustrated sample treatment assemblies 156 also support operation with a basket.

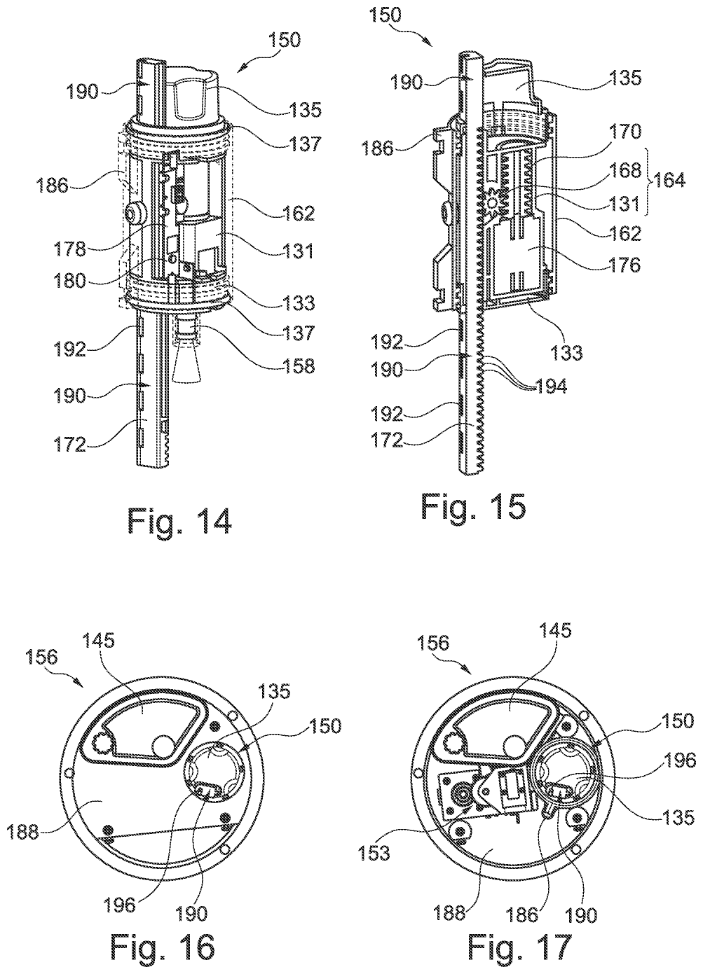

FIG. 13 illustrates a cross-sectional view of a sampling device 150 according to an exemplary embodiment of the present disclosure. FIG. 14 illustrates a three-dimensional view of the sampling device 150 of FIG. 13. FIG. 15 illustrates another cross-sectional view of the sampling device 150 of FIG. 13 and FIG. 14. FIG. 16 illustrates a plan view of the sampling device 150 of FIG. 13 to FIG. 15. FIG. 17 illustrates a transparent plan view of the sampling device 150 of FIG. 13 to FIG. 16.

As best seen in FIG. 13, the vertical movement of the sample taker 190 is driven via a rack and pinion assembly and is monitored via board mounted optical sensors 178. The optical sensors 178 detect gaps or slits 192 in the rib of the cannula rack 172. This allows a position, in particular a home position, an end stop position and a position corresponding to a removal of the cannula rack 172, to be monitored.

Still referring to FIG. 13, drive unit 176 provides a rotary drive force and is force coupled with worm drive gear 170 which transmits a drive force to pinion gear 168. Teeth of pinion gear 168 engage ribs 194 of cannula rack 172 which is thereby moved upwardly or downwardly while the rest of the sampling device 150 may remain spatially fixed in vertical direction 199. By integrating the described vertical motion mechanism 164 in the sampling device 150 (rather than in main unit 110), an individual vertical motion of the sample taker 190 of a certain sampling device 150 up and down is rendered possible independently of other sample treatment assemblies 156 of the dissolution apparatus 100. Thereby, individual sampling can be supported for each individual sample treatment assembly 156, and not all sample treatment assemblies 156 need to be necessarily sampled simultaneously. This increases the flexibility of using dissolution apparatus 100.

FIG. 17 illustrates a drive mechanism 153 for driving the stirring device 154. Electric energy for operating drive mechanism 153 may be supplied from main unit 110 of dissolution apparatus 100 by a cable connection and via cable adapter 143 (see FIG. 11).

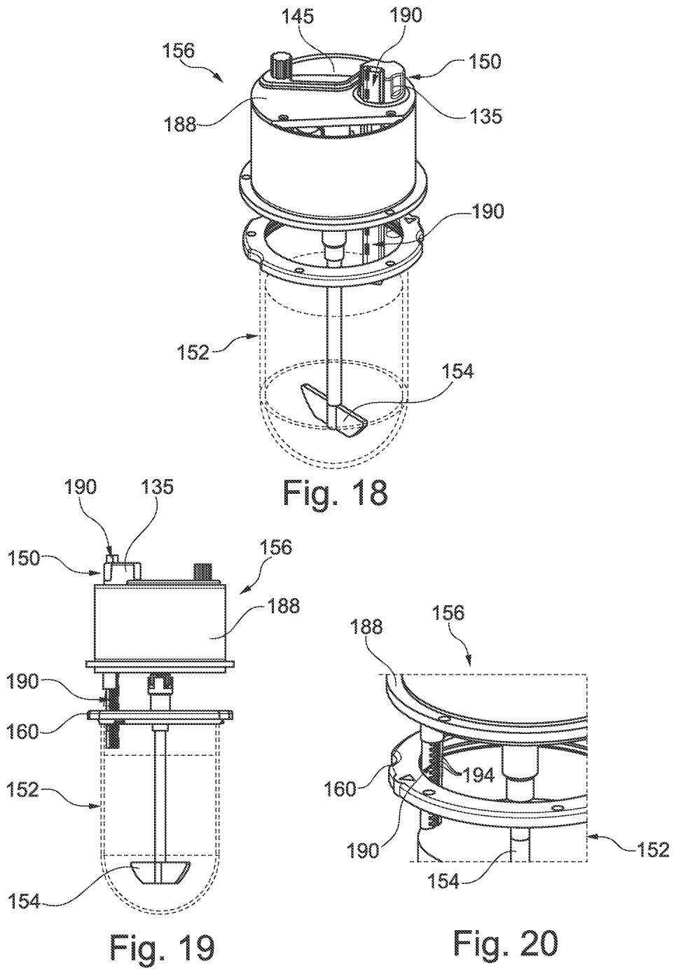

FIG. 18 illustrates a three-dimensional view of a sample treatment assembly 156 according to an exemplary embodiment of the present disclosure. FIG. 19 illustrates a side view of the sample treatment assembly 156 according to FIG. 18. FIG. 20 illustrates a detail of the sample treatment assembly 156 according to FIG. 18 and FIG. 19.

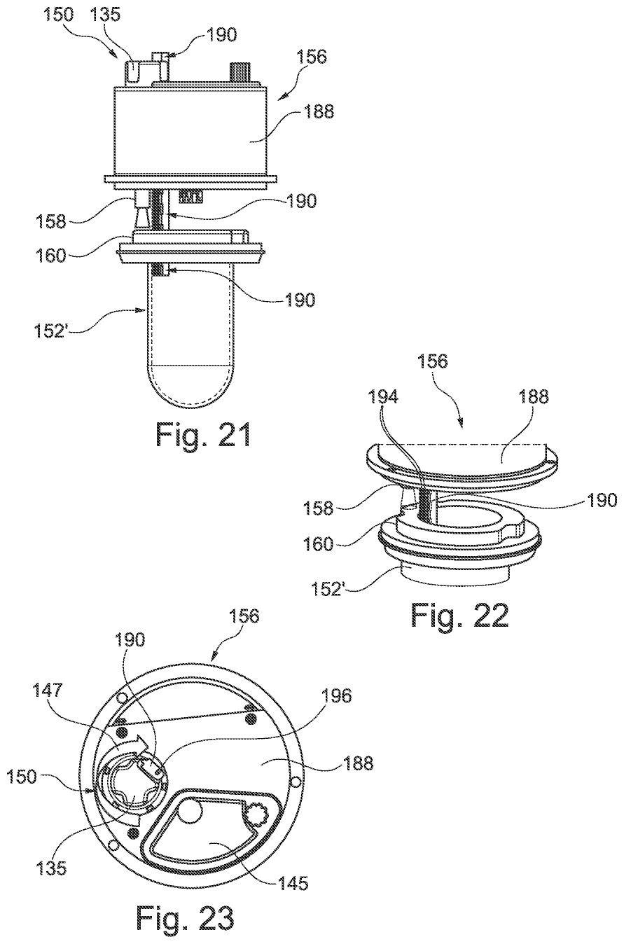

FIG. 21 illustrates a side view of a sample treatment assembly 156 with another vessel 152′ being smaller than the vessel 152 in FIG. 18 according to another exemplary embodiment of the present disclosure. FIG. 22 illustrates a detail of the sample treatment assembly 156 according to FIG. 21. FIG. 23 illustrates a plan view of the sample treatment assembly 156 according to FIG. 21 and FIG. 22.

Hence, FIG. 19 and FIG. 21 show different sample treatment assemblies 156 according to exemplary embodiments of the present disclosure using the same sampling device 150 for different vessels 152, 152′ of different dimensions. Using the same sampling device 150 for the different vessels 152, 152′ may be accomplished by adjusting a position of a sample taker 190 by rotation in a horizontal plane 198.

Vessel 152 according to FIG. 19 may for instance have a volume of 11. In contrast to this, vessel 152′ according to FIG. 21 has a volume of 250 ml. In order to use the same sampling device 150 with both vessels 152, 152′ and for arranging sample taker 190 at a correct radial position with respect to the respective vessel 152, 152′, sample taker 190 may be simply rotated horizontally between the different angular positions according to FIG. 19 and FIG. 21 so as to be arranged at different appropriate radial positions in relation to the respective vessel 152, 152′. Re-assembly of dissolution apparatus 100 for this change may be dispensable.

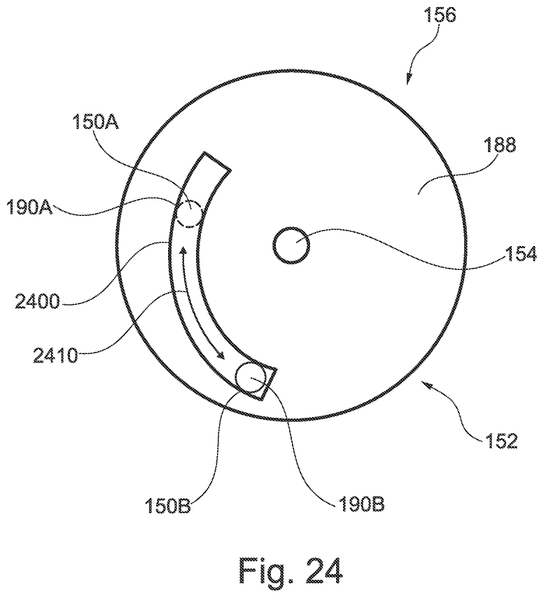

FIG. 24 schematically illustrates a different embodiment. The representation in FIG. 24 is similar to the representation in FIG. 9, however, reducing the features shown the only elaborate the specifics of that embodiment. The sample treatment assembly 156, also shown in plan view from top, allows to move the sample taker 190 along a guidance 2400. The guidance 2400 may be any kind of guiding mechanism allowing to move the sample taker 190 in a predefined path with respect to the vessel 152, in particular with respect to the central axis of the vessel 152, and can be a groove, recess, slot, opening, et cetera, preferably in the vessel cover member 188. However, any kind of mechanical fixation or attachment allowing to provide the guidance 2400 at or at least in respect to the mechanical shape of the vessel 152 is applicable accordingly. Even further, it is clear that any other kind of guidance allowing to guide the sample taker 192 to plural defined positions with respect to the vessel 152 can be applied as well. For example, an XY-mechanism, either fixed to the vessel 152 or at least provided with defined mechanical relation to the vessel 152, can be applied accordingly, for example using a grabbing mechanism, an arm, a handling arm, a turntable, robotics, et cetera.

In operation, as schematically depicted in FIG. 24, the sample taker 190 (maybe as part of the sampling device 150 or separated therefrom) may be moved from an initial position (indicated by reference numeral A) to a different position (indicated by reference numeral B) along the guidance 2400, as also indicated by the arrow 2410. For example, the sample taker 190 is moved from its initial position 190A into the position 190B.

In the exemplary embodiment of FIG. 24, the guidance 2400 is provided along an eccentric path around the central axis of the vessel 152 (which substantially corresponds to the position of the stirring device 154), so that the sample taker 190 can assume different positions within the plane of the central axis of the vessel 152, such as different radial positions with respect to the central axis of the vessel 152. It goes without saying that different shaping of the guidance 2400 may allow different locations of the sample taker 190.

The sample taker 190 is preferably part of the sampling device 150 as e.g. detailed with respect to FIGS. 2-30, but may also be separated therefrom, at least in the sense that the sample taker 190 is movable along the guidance 2400 independent from a movement of the sampling device 150. In the schematic representation of FIG. 24, the sample taker 190 and the sampling device 150 are depicted by the same symbol for the sake of simplicity.

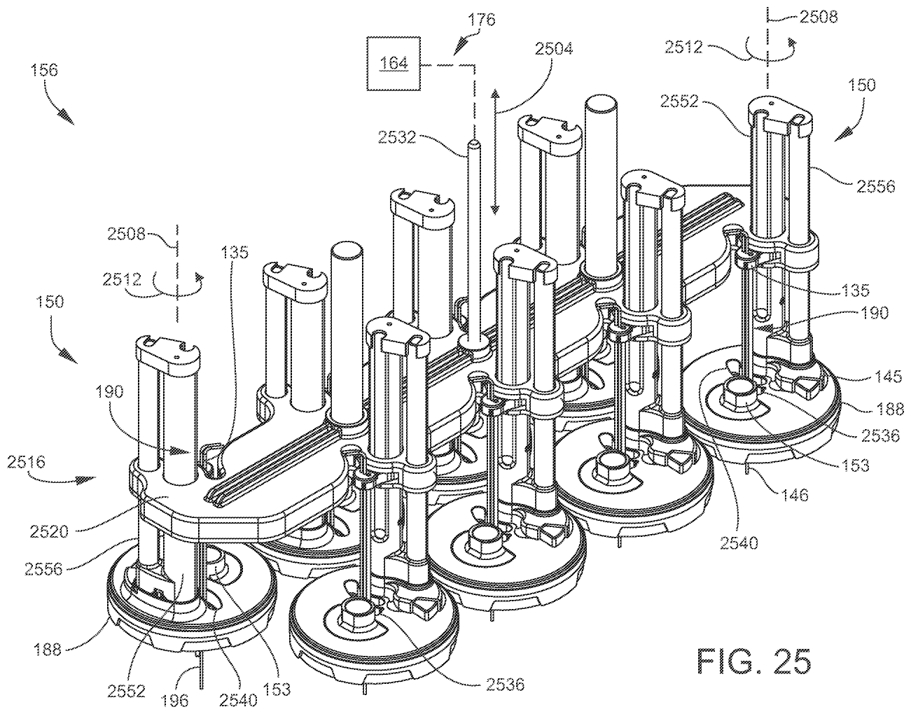

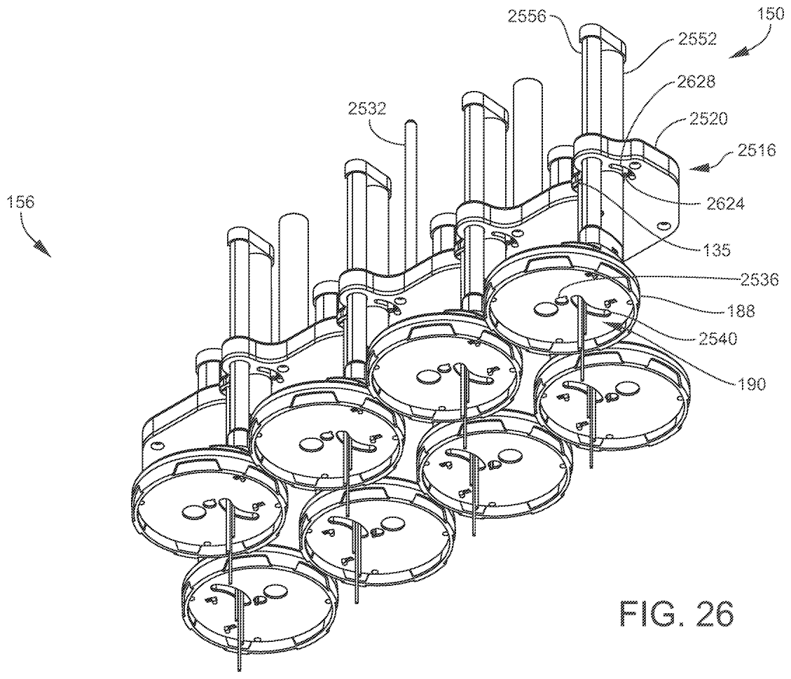

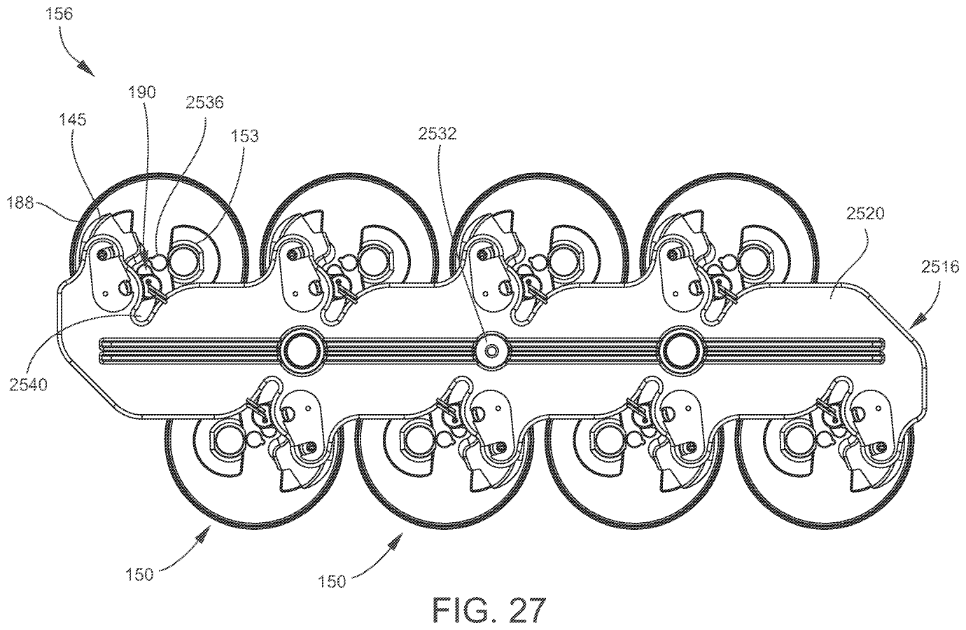

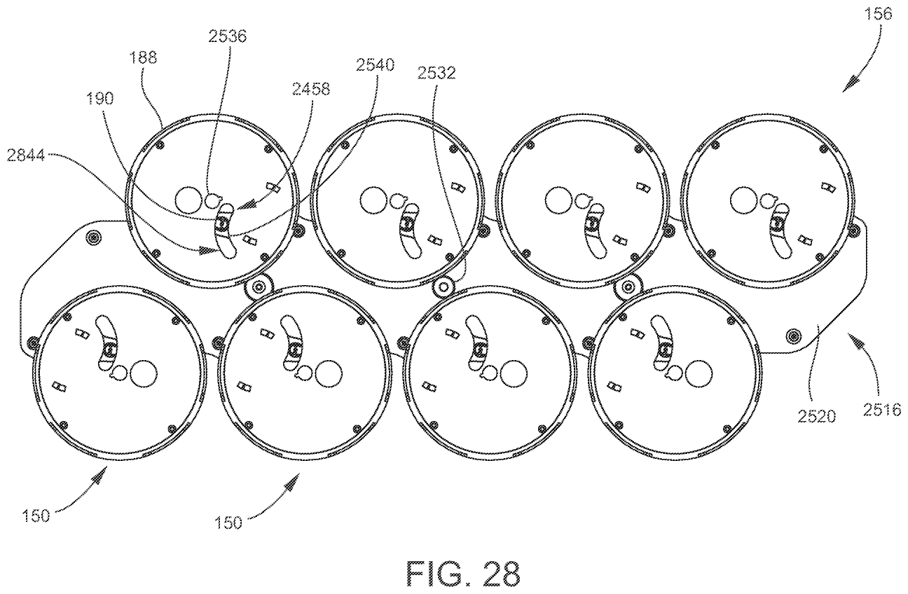

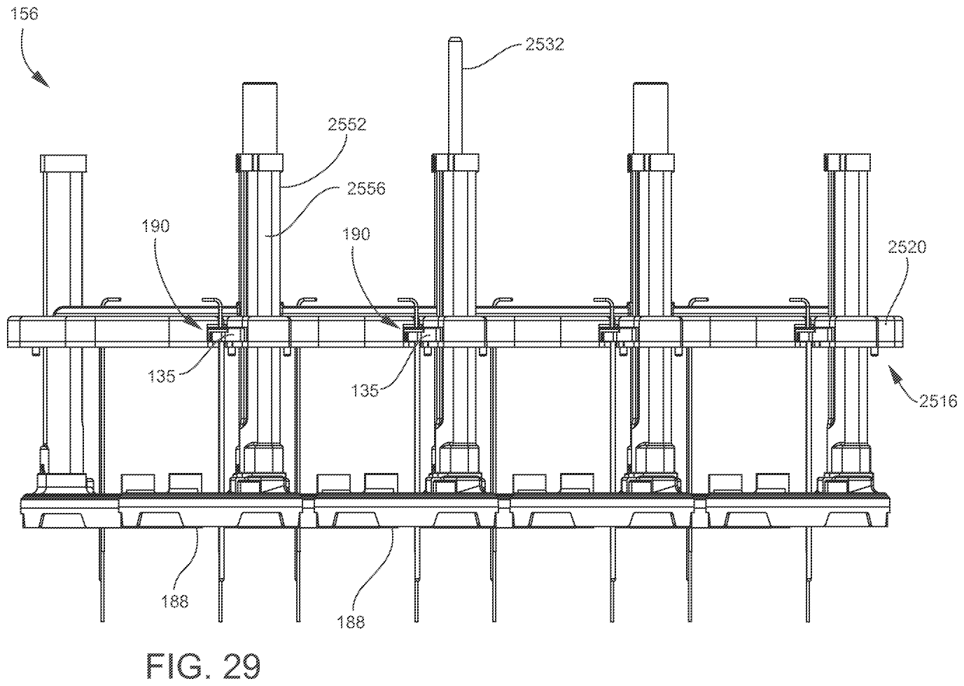

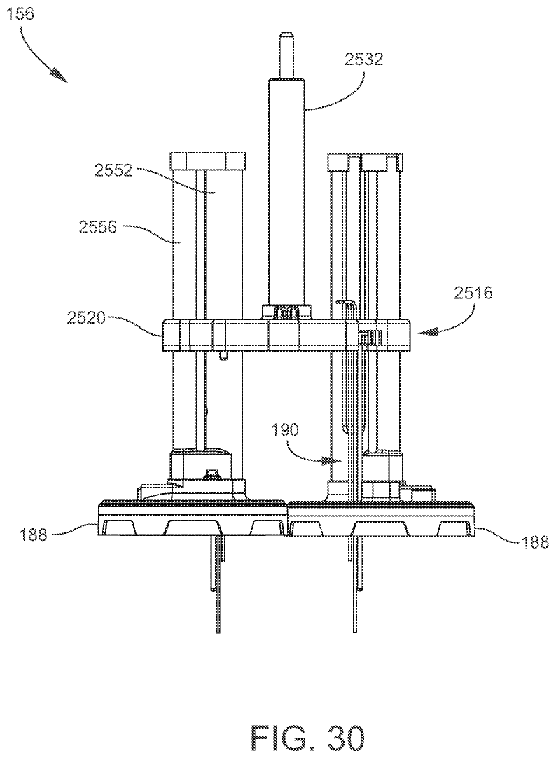

FIG. 25 illustrates a top perspective view of a sample treatment assembly 156 that includes a plurality of sampling devices 150 according to an exemplary embodiment of the present disclosure. FIG. 26 illustrates a bottom perspective view of the sample treatment assembly 156 according to FIG. 25. FIG. 27 illustrates a top plan view of the sample treatment assembly 156 according to FIG. 25. FIG. 28 illustrates a bottom top plan view of the sample treatment assembly 156 according to FIG. 25. FIG. 29 illustrates a front plan view of the sample treatment assembly according to FIG. 25. FIG. 30 illustrates an end plan view of the sample treatment assembly 156 according to FIG. 25.

The number of sampling devices 150 provided with the sample treatment assembly 156 may correspond to the number of vessels 152 mounted to a dissolution apparatus 100 (see, e.g., FIG. 1). That is, each sampling device 150 may be mounted at a corresponding vessel 152. Each sampling device 150 includes a corresponding sample taker 190 that may be or include a sampling cannula (or media aspirating cannula) 196 as described herein. Hence, each sample taker 190 may be inserted (lowered) into the corresponding vessel 152 (and immersed into media contained in that vessel 152), and may be removed (raised) from the corresponding vessel 152 (and out of media contained in that vessel 152), as depicted by a double-headed arrow 2504 in FIG. 25. At each vessel 152, the corresponding sample taker 190 is configured to be rotated about a sampling device axis 2508 parallel to a central axis of the vessel 152 (or offset from the central axis of the vessel 152 relative to the horizontal plane 198, see FIG. 1), as depicted by a few curved arrows 2512 in FIG. 25. This rotation of the sample taker 190 changes a radial distance of the sample taker 190 with respect to the central axis of the corresponding vessel 152.

In the present embodiment, the sample treatment assembly 156 includes a holding mechanism 2516 configured to support each sample taker 190 and accommodate rotation (or swiveling) of each sample taker 190 about the corresponding sampling device axis 2508. The holding mechanism 2516 may include a manifold 2520 that may be generally in the form of a plate or platform. The holding mechanism 2516 may further include, for each sampling device 150, an actuation element (or sampling arm) 135 at which a corresponding one of the sample takers 190 is mounted. The manifold 2520 may be at least partially hollow to accommodate the actuation elements 135. The actuation element 135 is movable relative to the holding mechanism 2516 such that actuation of the actuation element 135 rotates the sample taker 190 about the sampling device axis 2508 (i.e., along a curved travel path relative to the sampling device axis 2508) as depicted by the above-noted arrows 2512.

In the present embodiment, the actuation of the actuation elements 135 is manual (user-actuated). Hence, for example, a user may manipulate a selected actuation element 135 to rotate it. As shown in FIG. 26, manual actuation may be facilitated by user-grippable sliders 2624 that extend from the respective actuation elements 135 through respective curved through-holes 2628 formed in a bottom section (e.g., bottom plate) of the manifold 2520. The curvature of each through-hole 2628 corresponds to the curved travel path of the corresponding sample taker 190. Each curved through-hole 2628 accommodates the arcuate sliding of the corresponding slider 2624. In an embodiment, the ends of each curved through-hole 2628 may additionally serve as mechanical stops that limit the movement of the corresponding slider 2624. For example, the ends of each curved through-hole 2628 may define the ends of the range of angular positions to which the corresponding slider 2624 may be moved, thereby also defining the ends of the range of angular positions to which the corresponding sample taker 190 may be moved relative to the sampling device axis 2508.

In the present embodiment, the angular positions of the actuation elements 135 are individually adjustable. That is, each actuation element 135 is rotatable independently of the other actuation elements 135. In other words, the holding mechanism 2516 is configured to rotate each sample taker 190, via its corresponding actuation element 135, independently of other ones of the plurality of sample takers 190. In another embodiment, the holding mechanism 2516 may be configured such that all actuation elements 135 are actuated (actuatable) together simultaneously (i.e., in a synchronized manner). For example, all actuation elements 135 may be actuated by a single (common) slider, for example via an appropriate mechanical linkage between the actuation elements 135 and the single slider, as understood by persons skilled in the art. In other words, the holding mechanism 2516 may be configured to rotate all sample takers 190 together and simultaneously.

In another embodiment, the total number of actuation elements 135 (and associated sampling devices 150) provided with the sample treatment assembly 156 may be divided (at least conceptually) into groups (subsets) of actuation elements 135. For example, the illustrated total number of eight actuation elements 135 (for mounting at eight corresponding vessels 152) may be divided into two groups each containing four actuation elements 135, or four groups each containing two actuation elements 135, etc. In this case, the holding mechanism 2516 may be configured such that all actuation elements 135 of one group are actuated (actuatable) together simultaneously, but independently of the other group(s). In other words, the holding mechanism 2516 may be configured to rotate each sample taker 190 together with one or more of the other ones of the plurality of sample takers 190.

In the present embodiment, the actuation of the actuation elements 135 is manual as noted above. In another embodiment, the holding mechanism 2516 may be configured such that the actuation of the actuation elements 135 is automated (e.g., motorized) by providing appropriate components (e.g., motor(s), mechanical linkages, positional sensors, encoders, etc.) as understood by persons skilled in the art.

In the present embodiment, the holding mechanism 2516 (particularly the manifold 2520) is further configured to be movable along the vertical direction, and each sample taker 190 is movable with the holding mechanism 2516 along the vertical direction 199 (see FIG. 1) as depicted by the double-headed arrow 2504 in FIG. 25. Hence, movement of the holding mechanism 2516 in the vertical direction 199 raises or lowers the sample takers 190, particularly relative to the respective, underlying vessels 152. This vertical movement may be manual or automated (e.g., motor-driven). In the illustrated embodiment, the sample treatment assembly 156 includes a drive unit 176 configured to vertically move the holding mechanism 2516 and thus the sample takers 190. FIGS. 25-30 illustrate a vertically-oriented shaft (or arm, etc.) 2532 attached to the manifold 2520 that may be considered as being part of the holding mechanism 2516 or the drive unit 176. The drive unit 176 includes a motion mechanism 164 coupled to the shaft 2532. The motion mechanism 164 may schematically represent any assembly (or system) of components suitable for alternately raising and lowering the shaft 2532, and thus the manifold 2520 and sample takers 190, along the vertical direction 199 as depicted by the double-headed arrow 2504 in FIG. 25. Hence, the motion mechanism 164 may include, for example, a motor, gearing, transmission linkage, etc., as appreciated by persons skilled in the art. For control purposes, the motion mechanism 164 may communicate with an appropriate control unit (including, e.g., one or more computing devices, electronic circuitry, non-transitory computer-readable instructions, etc.) as appreciated by persons skilled in the art, all or part of which may be located in the main unit 110 shown in FIG. 1 and described above.

In another embodiment, the movement of the holding mechanism 2516 (e.g., manifold 2520 and sample takers 190) may be done manually. In this case, the shaft 2532 may be, or be coupled to, a user-grippable handle (or lever, etc.).

The sample treatment assembly 156 may further include a plurality of vessel cover members 188, for example, one for each sampling device 150. The vessel cover members 188 serve as lids that are mounted to the open tops of the corresponding vessels 152, as well as supports for other components of the sample treatment assembly 156, as described herein. Each vessel cover member 188 has a central hole 2536 through which a stirring device 154 extends as described herein. The central hole 2536 is located on, and thus corresponds to, the central axis of the vessel 152. Each vessel cover member 188 further has an arcuate (or curved) aperture 2540 through which the sample taker 190 is extendable into the vessel 152 when the sample taker 190 is mounted at the vessel 152. By operation of the actuation element 135, the sample taker 190 is movable within the arcuate aperture 2540 in the horizontal plane 198 while being rotated about the sampling device axis 2508. Each arcuate aperture 2540 accommodates the rotation of the sample taker 190 about the sampling device axis 2508. When the sample taker 190 is rotated, it follows a curved travel path corresponding to the curvature of the arcuate aperture 2540. The ends of the arcuate aperture 2540 may additionally serve as mechanical stops that limit the range of movement of the sample taker 190 (i.e., the range of angular positions relative to the sampling device axis 2508 attainable by the sample takers 190).

As noted above, rotation of the sample taker 190 changes the radial distance of the sample taker 190 relative to the central axis of the vessel 152. The sample taker may be rotated to different angular positions relative to the sampling device axis 2508, with each angular position corresponding to a different radial distance. For example, FIG. 28 shows two angular positions located at or near the two ends of the arcuate aperture 2540: a first angular position 2844 and a second angular position 2848. When moved to the first angular position 2844, the sample taker 190 is located at the maximum radial distance from the central axis of the vessel 152, which is useful for a larger (higher-volume) vessel 152 (e.g., having a volume of 1 L). When moved to the second angular position 2848, the sample taker 190 is located at the minimum radial distance from the central axis of the vessel 152, which is useful for a smaller (lower-volume) vessel 152 (e.g., having a volume of 250 mL). The sample taker 190 also may be operable at one or more intermediate angular positions between the first angular position 2844 and the second angular position 2848. The holding mechanism 2516 (e.g., manifold 2520 and/or actuation element 135) may be configured to provide discrete, fixed angular positions (including the first angular position 2844, second angular position 2848 and any intermediate angular positions) at which the sample taker 190 can be repeatably and accurately located and retained in place. For example, the sample taker 190 may “click” into any of the different angular positions provided, such as by utilizing spring-loaded balls and associated features (not shown) as may be provided by the manifold 2520 and/or actuation element 135.

In the present embodiment, for each sampling device 150, at least a part of a stirring device drive mechanism 153 for driving the rotation of (or additionally providing support for) the stirring device 154 as described herein may be mounted (or attached) to or integrated with the vessel cover member 188. Also in the present embodiment, for each sampling device 150, at least a part of a dosage delivery mechanism (e.g., a supply unit 145 for supplying media such as dosage units to the assigned vessel 152) as described herein may be mounted (or attached) to or integrated with the vessel cover member 188.

The sample treatment assembly 156 may further include, for each sampling device 150, one or more vertically-oriented pillars (i.e., may include at least one pillar) mounted (or attached) to or integrated with the vessel cover member 188 and arranged along respective pillar axes. Specifically in the illustrated embodiment, the sample treatment assembly 156 includes a first pillar 2552 and a second pillar 2556 located at each sampling device 150. In the present embodiment, the pillar axis of the first pillar 2552 corresponds to the sampling device axis 2508, such that the actuation element 135 and sample taker 190 are rotatable together around the (stationary) first pillar 2552. Each set of pillars (e.g., first pillar 2552 and second pillar 2556) passes through corresponding holes of the manifold 2520. By this configuration, the pillars may assist in guiding the vertical motion of the manifold 2520 (and thus the actuation elements 135 and sample takers 190) relative to the (stationary) pillars. Moreover, the use of at least two pillars for each sampling device 150 may enhance stability and positional accuracy. For example, the pillars may ensure that the vessel cover members 188 always remain oriented in the horizontal plane 198 so that they always align correctly with the underlying vessels 152.

In some embodiments, the first pillars 2552 and the second pillars 2556 may be configured to provide additional functions. For example, each first pillar 2552 may be configured to accommodate (support, guide, etc.) a fiber-optic measurement probe. Each second pillar 2556 may be configured to be part of or accommodate the dosage delivery mechanism. For example, each second pillar 2556 may contain a servo drive harness for the corresponding dosage delivery mechanism.

In the illustrated embodiment, the manifold 2520 of the holding mechanism 2516 is common to all sampling devices 150 such that all sample takers 190 are moved together (i.e., in a synchronized manner) along the vertical direction. In another embodiment, the holding mechanism 2516 may be configured to move each sample taker 190 (e.g., selectively) along the vertical direction independently of other sample takers 190. For example, instead of providing a single (common) manifold 2520 supporting all sample takers 190 together, the holding mechanism 2516 may provide a plurality of support plates (e.g., individual manifolds), with each support plate being associated with one sampling device 150 and associated sample taker 190 and also a corresponding actuation element 135. In this case, the above-noted drive unit 176 and associated components may be modified to realize individual/independent actuation of and control over the vertical movement of the sample takers 190.

As another alternative, the sampling devices 150 may be divided into groups, with each group containing two of more of the sampling devices 150 and their associated components (sample taker 190, actuation element 135, etc.). For example, the illustrated total number of eight sampling devices 150 may be divided into two groups of four sampling devices 150, four groups of two sampling devices 150, etc. In this case, each group of sampling devices 150 may be supported by a single (common) support plate separately from other groups. Thus, for example, the holding mechanism 2516 may include two such support plates, or four such supports plates, etc., depending on the number of sampling devices 150 contained in each group. By this configuration, the vertical motion of different groups of sample takers 190 may be performed independently of the other groups. In other words, the holding mechanism 2516 may be configured to move each sample taker 190 along the vertical direction together with one or more of the other sample takers 190, depending of the number of sample takers 190 assigned to that particular group of sampling devices 150/sample takers 190. In this case, the above-noted drive unit 176 and associated components may be modified to realize individual/independent actuation of and control over the vertical movement of the pre-designated groups of sampling devices 150/sample takers 190.

It should be noted that the term “comprising” does not exclude other elements or features and the term “a” or “an” does not exclude a plurality. Also elements described in association with different embodiments may be combined. It should also be noted that reference signs in the claims shall not be construed as limiting the scope of the claims.

What is claimed is:

1. A sampling device for taking sample from a vessel of a dissolution apparatus, wherein the sampling device is configured to be mounted at the vessel and comprises a sample taker configured to be movable within a horizontal plane when mounted at the vessel.

2. The sampling device according to claim 1, wherein the sample taker is configured to be rotatable within the horizontal plane.

3. The sampling device according to claim 1, wherein the sample taker is configured to be rotatable about a central axis of the sampling device.

4. The sampling device according to claim 1, comprising at least one of the following features:
wherein the sample taker is configured to be movable along a vertical direction when mounted at the vessel;
wherein the sample taker comprises a sampling cannula for taking sample;
wherein the sample taker is configured for withdrawing sample from the vessel.

5. The sampling device according to claim 1, comprising an antenna configured for wireless communication with a transponder of the vessel.

6. The sampling device according to claim 1, comprising a manual actuation element configured for being manually actuated by a user for manually moving the sample taker within the horizontal plane.

7. The sampling device according to claim 1, comprising one of the following features:
at least one sensor configured for sensing sensor data indicative of a position and/or a motion of the sample taker;
at least one sensor configured for sensing sensor data indicative of a position and/or a motion of the sample taker, wherein the sample taker comprises at least one marker to be sensed by the at least one sensor;
at least one sensor configured for sensing sensor data indicative of a position and/or a motion of the sample taker, wherein the sample taker comprises at least one slit to be sensed by the at least one sensor.

8. The sampling device according to claim 1, comprising a motion mechanism configured for vertically moving the sample taker.

9. The sampling device according to claim 8, wherein the motion mechanism comprises a pinion gear cooperating with a worm drive gear for engaging an array of ribs of a cannula rack of the sample taker.

10. The sampling device according to claim 1, comprising a tubular sheath accommodating at least part of a motion mechanism for moving the sample taker along a vertical direction.

11. A sample treatment assembly for a dissolution apparatus, the sample treatment assembly comprising:
the sampling device according to claim 1; and
the vessel for accommodating the sample, wherein the sampling device is mounted at the vessel.

12. The sample treatment assembly according to claim 11, wherein the sampling device is laterally displaced with respect to a central axis of the vessel so that rotation of the sample taker changes a radial distance of the sample taker with respect to the central axis of the vessel.

13. The sample treatment assembly according to claim 11, comprising a stirring device for stirring sample and being mounted at the vessel.

14. The sample treatment assembly according to claim 13, comprising at least one of the following features:
wherein the stirring device comprises a paddle;
wherein the stirring device extends along a central axis of the vessel.

15. The sample treatment assembly according to claim 11, comprising a vessel cover member covering an opening of the vessel and accommodating the sampling device, in a horizontally rotatable way, for mounting the sampling device at the vessel.

16. A dissolution apparatus for testing dissolution of a sample, wherein the dissolution apparatus comprises the sampling device according to claim 1.

17. The dissolution apparatus according to the claim 16, comprising a plurality of sampling devices, wherein at least one of the sampling devices comprises the sample taker that is configured to be movable within the horizontal plane and/or along a vertical direction independently of a sample taker of at least one other of the sampling devices.

18. A dissolution apparatus for testing dissolution of a sample, comprising:
a vessel for accommodating sample;
a sampling device comprising a sample taker for taking sample from the vessel; and
a vessel cover member covering an opening of the vessel and accommodating the sampling device for mounting the sampling device at the vessel,
wherein the sample taker is configured to be rotatable in a concentric way about an axis of the sampling device and in an eccentric way about an axis of the vessel.

19. A method of operating a dissolution apparatus for taking sample from a vessel, the method comprising:
mounting a sampling device at the vessel accommodating the sample; and
moving a sample taker of the sampling device within a horizontal plane and relative to the vessel when mounted at the vessel.

20. The sampling device according to claim 1, wherein the sample taker is configured to be rotated about a sampling device axis parallel to a central axis of the vessel, and rotation of the sample taker changes a radial distance of the sample taker with respect to the central axis of the vessel.
